(19) United States
(12) Patent Application Publication
(10) Pub. No.: US 2012/0041006 A1
(43) Pub. Date: Feb. 16, 2012

(54) Compositions for Raising Uric Acid Levels and Methods of Using the Same

(76) Inventors: Meir S. Sacks, Pittsburgh, PA (US); Knox Van Dyke, Morgantown, WV (US)

(21) Appl. No.: 13/016,356

(22) Filed: Jan. 28, 2011

Related U.S. Application Data
(63) Continuation of application No. 10/804,760, filed on Mar. 19, 2004, now abandoned.
Application No. 10/804,760 is a division of application No. 09/981,222, filed on Oct. 16, 2001, now abandoned.
Application No. 09/981,222 is a continuation-in-part of application No. 09/449,037, filed on Nov. 24, 1999, now abandoned.
Application No. 09/449,037 is a continuation-in-part of application No. 09/449,161, filed on Nov. 24, 1999, now abandoned.
Application No. 09/449,037 is a continuation of application No. 09/127,184, filed on Jul. 31, 1998, now abandoned.
Application No. 09/449,161 is a continuation of application No. 09/127,184, filed on Jul. 31, 1998, now abandoned.

(51) Int. Cl. A61K 31/522; A61P 7/00

(52) U.S. Cl. 514/263.33

(57) ABSTRACT

Compositions for the treatment of uric acid deficiency are disclosed. The compositions generally comprise either a precursor or derivative of uric acid, which, when administered to a patient, will result in a raising of the uric acid levels in that patient. The compositions can optionally comprise one or more additional active ingredients such as antioxidants, glutathione precursors, or inhibitors of NO synthase or homocysteine. Methods for raising uric acid levels in a patient are also disclosed. These methods are useful for in the treatment of various illnesses, such as cancer, infectious disease, Alzheimer disease and neurodegenerative diseases. Use of improved solutions comprising the present compositions in organ preservation is also disclosed.

### CROSS REFERENCE TO RELATED APPLICATIONS

This application is a continuation of U.S. application Ser. No. 10/804,760 filed Mar. 19, 2004, which is a divisional of U.S. application Ser. No. 09/981,222 filed Oct. 16, 2001, which is a continuation-in-part of U.S. application Ser. No. 09/449,037 filed Nov. 24, 1999 and a continuation-in-part of U.S. application Ser. No. 09/449,161 filed Nov. 24, 1999, both of which are continuations of U.S. application Ser. No. 09/127,184 filed Jul. 31, 1998, all of which are incorporated herein by reference.

### FIELD OF THE INVENTION

The present invention relates to compositions comprising one or more uric acid precursors or uric acid derivatives. The compositions are useful in the treatment of diseases in which low levels of uric acid are observed. Accordingly, methods for treating such diseases are also within the scope of the present invention.

### BACKGROUND INFORMATION

Oxidative damage is believed to be a mechanism of damage in many diseases. Such damage is found, for example, in diseases such as cancer, rheumatoid arthritis, heart disease, cataracts, inflammatory diseases, artery occlusion, diabetes, neurodegenerative diseases, and age-related macular degeneration. Free radicals, a major cause of oxidative damage, may be generated by environmental radiation, air pollution, inflammation and excessive physical and mental exertion. A free radical is an atomic species having a free electron, and is typically propagated from oxygen or nitric oxide or by specific enzymatic reactions like NADPH oxidase, xanthine oxidase and NO synthase I, II or III. Peroxynitrite (OONO)⁻ is a strong oxidizer formed from superoxide [.O]⁻ and nitric oxide [.NO], which, among other things, causes tissue damage and damage to membrane lipids, DNA and RNA of cells. Peroxynitrite has 1,000 times the oxidative activity as concentration-equivalent amounts of hydrogen peroxide, and is therefore a potent oxidizer capable of causing significant damage in vivo.

Free radical damage is believed to be caused when an oxygen atom acquires a free electron to become a free radical; radicals combine to become strong oxidants which can cause oxidative damage. Free radicals attach to molecules in the body resulting in changes to the endogenous molecules' normal function; in this manner, the structure and function of the molecule changes. Nucleic acids, proteins, enzymes and lipid molecules are all susceptible to oxidation. Lipid oxidation can cause damage to membrane systems including cell membranes, membranes of cellular organelles, and other membranes. Protein oxidation can lead to cell structure damage. Enzymatic oxidation can result in changes in metabolic rates. Nucleic acid damage can lead to cell mutation and cell necrosis.

The damage caused by oxidants formed from free radicals is alleviated, at least in part, by various protective cellular mechanisms, such as antioxidants and radical scavengers in both the membrane lipids (for example, α-tocopherol and β-carotene) and aqueous (for example, glutathione and ascorbic acid) phases of cells, as well as enzymes such as superoxide dismutase and catalase. Uric acid has also been shown to be an excellent free radical protective factor or antioxidant. Individuals having a low uric acid blood concentration, therefore, are less able to mount a sufficient antioxidant defense against free radicals and oxidative damage. As such, individuals having an illness or a condition in which uric acid levels are below normal (i.e., below about 4.9 mg per 100 ml of blood) may experience the degeneration that accompanies oxidation or free radical attack. Low uric acid results in lack of protection against oxidants or free radicals that originate in diseased states or that are caused by environmental factors.

### SUMMARY OF THE INVENTION

The present invention relates to compositions comprising one or more uric acid precursors or uric acid derivatives, as those terms are defined herein. The present compositions can further include one or more of an additional antioxidant, a precursor of glutathione, an inhibitor of homocysteine formation, and an inhibitor of nitric oxide synthase.

The present invention is also directed to a method for raising uric acid levels in a patient whose uric acid levels are below normal; i.e., below about 4.9 mg/100 ml of blood. This method is particularly applicable in the treatment or prevention of various illnesses, especially those in which oxidative damage occurs. The methods generally involve the administration of an effective amount of the present compositions.

The present invention is therefore directed to compositions and methods for increasing uric acid levels in a patient. Typically, the patient will be afflicted with an illness in which base levels of uric acid are depleted. By maintaining uric acid levels at or above normal, oxidative damage is minimized, if not eliminated, according to the present invention.

The present invention is also directed to methods for using the present compositions to preserve organs that have been harvested for transplant.

It is therefore an aspect of the invention to provide compositions that can raise uric acid levels.

Another aspect of the invention is to provide such compositions for the study and treatment of diseases and disorders in which low uric acid levels are present.

These and other aspects of the invention will be apparent based upon the following description and appended claims.

### DETAILED DESCRIPTION

The present invention is directed to compositions comprising one or more uric acid precursors or uric acid derivatives. The term “uric acid precursor” as used herein refers generally to any compound that will metabolize in the body to become uric acid or a molecule that is structurally and/or functionally equivalent to uric acid. This equivalence can be determined by exposing the molecule to oxidizing agents such as peroxynitrite, hypochlorite or peroxyhypochlorite and comparing the response of the molecule to that of uric acid. A comparable response indicates structural and/or functional similarity. Particular examples of uric acid precursors include but are not limited to hypoxanthine, xanthine, inosine, derivatives of these compounds and biological equivalents thereof. Derivatives of these compounds include precursors that have been modified to increase their solubility and/or bioavailability, such as alkylated derivatives, sugar derivatives and salt derivatives. Biological equivalents of these compounds include those which, when put into the body, are metabolized by purine synthesis into uric acid. The determination of equivalence can therefore be determined by one skilled in the art by measuring the level of uric acid in the blood both before and after administration of the compound.

“Uric acid derivative” as used herein refers to uric acid, or any of its precursors as described above, that have been modified to increase their solubility. Examples include alkylated derivatives, sugar derivatives and salt derivatives of uric acid. Alkylated derivatives include uric acid, or structurally and/or functional equivalent molecules, to which one or more alkyl groups is chemically attached. The alkyl groups can have between one and twenty carbons; methyl groups are particularly suitable. Sugar derivatives, generally referred to as “osine” compounds, include uric acid, a precursor or equivalents thereof to which a sugar moiety is chemically attached. Any sugar can be used according to the present invention, provided it can be attached to the uric acid molecule. Examples include ribose and deoxyribose sugars. Examples of sugar derivatives suitable for use in the present invention therefore include xanthosine, which is a sugar derivative of the precursor xanthine, and uric acid osine, which is a sugar derivative of uric acid itself. Similarly, salt derivatives of uric acid include uric acid, an equivalent or a precursor molecule to which is attached one or more pharmaceutically acceptable salts. Examples include sodium, potassium, calcium, lithium and ammonium salts. These can be prepared using the appropriate base warming reaction, known to those skilled in the art.

It will be appreciated by those skilled in the art that uric acid is relatively insoluble. As such, uric acid has poor bioavailability. The addition of an alkyl moiety, carbohydrate moiety, or the salt moiety increases the solubility of the molecule. The present uric acid derivatives can more easily cross the cell membrane than can uric acid alone. Once in the cell, the uric acid derivative converts to uric acid and the sugar, salt or other moiety used in its formation. In this manner, uric acid is effectively delivered to cells, and blood plasma uric acid concentrations are increased. Use of the present uric acid derivatives is therefore much more efficient in raising blood plasma uric acid levels than is the administration of uric acid itself. Moreover, administration of uric acid alone can result in elevated uric acid blood levels that can lead to gouty conditions. Also, high levels of uric acid can cause kidney toxicity and even kidney failure. For the same reasons, the administration of a uric acid precursor is preferable to uric acid itself, since the precursors will result in an increase in the uric acid levels without the attendant shortcomings of the administration of uric acid alone. The present invention addresses these issues, as the uric acid precursors and derivatives described herein are not toxic to the patient. In the case of uric acid precursors, they are present in the body and the present methods serve to increase these levels.

The compositions of the present invention can further comprise one or more additional antioxidants. It will be appreciated that the presence of an additional antioxidant will further serve to scavenge free radicals and oxidants, and therefore minimize oxidative damage in a patient. Any antioxidant can be used according to the present invention. Examples include vitamin E, vitamin C and its derivatives such as ester C (the calcium salt of vitamin C), dehydro-L-ascorbate C (an oxidized derivative of vitamin C), ester C of dehydro-L-ascorbate (an oxidized derivative of ester C) and lipidated derivatives such as ascorbic acid palmitate; these compounds are collectively referred to herein as vitamin C derivatives. Other antioxidants include polyphenols and cysteine derivatives. This list is not meant to be exhaustive.

If the compositions include vitamin C, which is water soluble, it is also desirable to include a compound that assists in the uptake of the vitamin by the cells; examples include polyphenols, tannins and epigallocatechin gallate (EGCg). Similarly, if vitamin E, which is fat soluble, is used in the present compositions, coenzyme Q10 can be added as an additional fat soluble antioxidant to assist in efficacy of the vitamin.

The compositions of the present invention can also optionally comprise one or more inhibitors of homocysteine formulation, and/or inhibitors of NO synthase. Inhibitors of homocysteine include, for example, vitamin B6 and folic acid; inhibitors of NO synthase include anti-inflammatory steroids such as prednisone and L-NAME.

The present compositions can further comprise precursors of glutathione. Glutathione is an antioxidant, and is therefore also useful in the reduction of oxidative damage. N-acetyl-1-cysteine is a suitable glutathione precursor. Because n-acetyl-1-cysteine makes both glutamoyl cysteine and glutathione, if used in the present composition it should be used in conjunction with compounds that inhibit the formation of homocysteine. Homocysteine has been linked to heart disease, and it would therefore be undesirable to increase levels of this amino acid.

More than one additional antioxidant, precursor of glutathione, inhibitor of NO synthase or inhibitor of homocysteine can be used in the present invention.

It is especially advantageous to formulate compositions in dosage unit form for ease of administration and uniformity of dosage. Dosage unit form as used herein refers to physically discrete units suited as unitary dosages for the patient to be treated, each unit containing a predetermined quantity of the active ingredient(s), or “effective amount” calculated to produce the desired effect. The specification for the dosage unit forms of the invention are dictated by and directly dependent on the characteristics of the active ingredients, the particular therapeutic effect to be achieved, and the limitations inherent in the art of compounding such active ingredients for the treatment of sensitivity in individual patients.

For example, the dosage form should contain an amount of uric acid precursor or derivative effective to raise uric acid levels. Typically, this will be an amount effective to raise levels of uric acid to above about 4.9 mg of uric acid per 100 ml of blood, and will be sufficient to maintain the patient's uric acid levels between about 4.9 mg and 10.0 mg of uric acid per 100 ml of blood. It will be appreciated that uric acid levels above about 8.0 can cause gout in a normal individual. The individuals treated according to the present invention can tolerate higher uric acid levels because of their diminished ability to produce and/or maintain uric acid. Typically, this effective amount will be between about 100 mg and 25 g per dosage, such as between about 1 and 20 g, or between about 2 and 10 g.

Any additional antioxidant, if used, should be present in an amount that will effect the desired level of oxidative protection in the patient, and can be determined by one skilled in the art such as by using cellular assays. If, for example, vitamin C or a derivative thereof is used, an effective dosage will typically be about 1-5 g, whereas if vitamin E is used, between 1,000 and 3,000 IU could be used.

Enough glutathione precursor should be used to produce the desired amount of glutathione in the patient. In dosage form, this will typically be between about 500 and 2,000 mg. Similarly, a sufficient amount of homocysteine inhibitor should be used, and will typically be higher if n-acetyl-l-cysteine is used as the glutathione precursor. One skilled in the art can determine the appropriate amount of homocysteine inhibitor, based on the amount of the other ingredients in the composition.

The present invention is therefore also directed to a pharmaceutical composition comprising at least one uric acid precursor or uric acid derivative and one or more additional antioxidants, glutathione precursors, NO synthase inhibitors, or homocysteine inhibitors. The active ingredients of the present compositions are preferably contained in a pharmaceutically acceptable carrier. “Pharmaceutically acceptable carrier” includes any and all solvents, dispersion media, coatings, antibacterial and antifungal agents, isotonic and absorption-delaying agents, and the like. The use of such media and agents for pharmaceutically active substances is well known in the art. Use of any of these media or agents is contemplated for use in the compositions of the present invention, absent compatibility problems with the active compound. Vehicles or carriers standardly used in the pharmaceutical arts for the administration of amino and nucleic acids and antioxidants can be adapted for use in the present invention by one skilled in the art. The pharmaceutical compositions can be formulated for oral, sublingual, transdermal, intravenous, anal or topical administration, with the oral and sublingual routes being most typical.

The present invention is also directed to a single oral dose of a uric acid derivative or uric acid precursor effective to raise uric acid levels in a human. This effective amount is as noted above for the dosage unit form.

The present invention is also directed to a method for raising uric acid levels in a patient, and, once raised, maintaining those levels within an acceptable range. Typically, uric acid levels should be at least about 4.9 mg per 100 ml blood. Uric acid levels within the range of 4.9 to 10.0 mg per 100 ml of blood are desired in patients with impaired ability to produce and/or maintain uric acid. Accordingly, the present method comprises the step of administering to a patient an effective amount of at least one uric acid derivative or uric acid precursor so as to bring/maintain the patient's uric acid level to within the desired range. In this manner, treatment of a disease state in a patient is effected.

In addition to the administration of one or more uric acid precursors/derivatives, the present methods further comprise administration of effective amounts of one or more additional antioxidants, precursors of glutathione or inhibitors of NO synthase or homocysteine. Administration of all of these components can be either concurrent or sequential.

“Treatment” is intended to encompass both therapeutic and prophylactic treatment of any of the illnesses or disease states discussed below. For ease of reference, “therapeutic benefit” and “therapeutic effect” are therefore used collectively to refer to a benefit that is either therapeutic or prophylactic; this includes treatment to maintain uric acid at the desired levels. A number of therapeutic benefits can be achieved according to the present methods. For example, administration of the present compounds can slow down or even stop the disease-mediated damage, alleviate symptoms of the disease, and the like.

The term “illness” or “disease state” as used herein refers generally to any illness or disease state in which a patient's uric acid level is below about 4.9 mg per 100 ml blood. Examples include cancer, rheumatoid arthritis, inflammatory diseases, infectious diseases, lung disease, neurodegenerative diseases, heart disease, artery occlusion, immunological disease, macular degeneration, Alzheimer's disease and diabetes. Neurodegenerative diseases can include, for example, Alzheimer's disease, aging, Parkinson's disease, multiple sclerosis, ALS, and the like.

“Patient” is used herein to refer to members of the animal kingdom, including but not limited to humans. Patients particularly suitable for treatment according to the present methods include those whose uric acid levels are below about 4.9 mg per 100 ml of blood.

“Effective amount,” as used herein in reference to the present treatment methods, refers to that amount of the present compositions needed to bring about the desired effect in a patient. Most typically, an effective amount will be that amount that results in raising uric acid levels in vivo to within the range discussed above. Whether suitable uric acid levels have been achieved can be determined by uric acid analysis, either enzymatic or non-enzymatic. The performance of these tests is well within the skill of those practicing in the art. The effective amount will vary depending on various factors including the patient to be treated, the illness being treated, the severity of the illness, the patient's reaction to the treatment and the like. The determination as to what is an effective amount for each patient is within the skill of those practicing in the art, and can be guided by objective measurements such as levels of uric acid, levels of antioxidant, levels of homocysteine, levels of glutathione and levels of NO synthase in the blood. An effective amount of uric acid precursor or derivative will typically be between 100 mg and 25 g per clay, such as 1-20 g per day or 2-10 g per day. An effective amount of antioxidant, if used, will typically be two to three times the recommended daily amount for each compound. Similarly, the amount of homocysteine inhibitor, if used, should be about two to three times the recommended daily amount. If n-acetyl-1-cysteine is used as the glutathione precursor, it should be given in an amount of about 500 and 2,000 mg per day. Finally, if an NO synthase inhibitor is used, such as prednisone, it should be administered in an amount of about 20-60 mg/day and is preferably administered only two to three times a week.

The present invention further relates to the preservation of biological materials for transplantation, and more particularly to compositions and methods for the resurrection and preservation of organs, tissues and cells from mammals.

When transplant organs are removed from the donor's body, the blood supply is interrupted. This action also interrupts the source of the organ's supply of oxygen, carbon dioxide, nitric oxide and nutrition, as well as the liquids that contain the necessary salts to create the correct osmotic pressure for a healthy osmotic environment for the tissue. Organ preservation methods are directed at minimizing the effects of interrupting the blood supply.

The composition commonly known as the University of Wisconsin Solution, the formula for which is set forth below, is a common solution used for the preservation of harvested organs. The original Wisconsin Organ Preservation Solution has allowed preservation of a variety of organs for transplantation including heart, liver, kidney and lungs. That solution typically comprises:
5% hydroxyethyl starch having a molecular weight of from about 200,000 to about 300,000 and a degree of substitution of from 0.4 to 0.7
25 mM KH₂PO₄
3 mM glutathione
5 mM adenosine
A0 mM glucose
10 mM HEPES Buffer (Sigma Chemical Company)
5 mM magnesium gluconate
1.5 mM CaCl₂
105 mM sodium gluconate
200,000 units of penicillin
40 units insulin
16 mg Dexamethasone
12 mg Phenol Red
pH 7.4-7.5

This solution has found widespread clinical application for the preservation of the major intra-abdominal organs, and is the subject of three issued U.S. patents (U.S. Pat. No. 4,798,824; U.S. Pat. No. 4,873,230; U.S. Pat. No. 4,879,283), all of which are incorporated herein by reference as if set forth in their entirety herein.

The present invention provides improved compositions for the preservation of biological materials, which compositions are formulated to reduce or eliminate reperfusion injury (“RI”) and/or to decrease antigenic response in a recipient upon transplantation. RI and antigenic response are two of the major causes of organ rejection. Generally, the compositions comprise Wisconsin Solution to which has been added a uric acid precursor or derivative and optionally one or more of the other components discussed herein, including additional antioxidants, inhibitors of homocysteine formulation, inhibitors of NO synthase and precursors of glutathione.

An improved Wisconsin Solution is disclosed wherein the improvement comprises the addition to the typical Wisconsin Solution of an effective amount of a uric acid derivative or precursor, as those terms are described above. The improved Wisconsin Solution can further comprise one or more of additional antioxidants, inhibitors of homocysteine formation, inhibitors of NO synthase and/or precursors of glutathione. Again, these compounds are as described above.

The improved preservation compositions of the present invention provide for the resurrection and preservation of transplantable organs, which compositions reduce or eliminate R1, and increase organ viability for extended periods of time. The compositions also reduce antigenic response in a recipient following transplantation. In addition to the components discussed above, the improved compositions or solutions preferably contain a significant amount of a water soluble substance to inhibit Nf kappa b. The improved solutions may also contain a large amount of non-assimilated polymer that has the ability to bind fat soluble substances that themselves might not be readily soluble. The improved compositions of the present invention may also contain L-arginine, and/or an equivalent nitric oxide (NO) donor and/or a substrate for NO. Soluble xanthine oxidase inhibitor may also be provided.

Nitric oxide and superoxide anion can be toxic to the transplantable biological material, but their respective production can be blocked via the inhibition of NO synthase or NADPH oxidase or xanthine oxidase, or through the activation of any of these enzymes. Substances that can either intercept nitric oxide and/or superoxide anion or react with peroxynitrite would prevent or at least minimize damage from occurring. A water soluble spin label, such as TEMPO or 4-hydroxyTEMPO, is suggested due to its properties as a recyclable superoxide dismutase mimic, to react with superoxide and convert it into hydrogen peroxide. In addition, an inhibitor/binder of, or reactant with, nitric oxide (NO) can also be utilized to lower the amount of nitric oxide present so peroxynitrite cannot be formed. It is contemplated that ascorbic acid, or polyphenols (e.g. those isolated from green tea), and N-acetyl cysteine could be used as inhibitor/binders of NO. Ascorbic acid and polyphenols are known to destroy peroxynitrite, and N-acetyl cysteine is a superior producer of L-glutathione.

Yet additional components may be included in the present improved solutions which: maintain a desired pH; inhibit peroxynitrite; serve as a source of magnesium; inhibit nitric oxide synthase; provide anti-bacterial action against gram positive and gram negative bacteria; provide potassium and phosphate to balance the osmolarity of the solution; react intracellularly with superoxide anion to form hydrogen peroxide; serve as a backup energy source; provide essential amino acids; allow glucose to penetrate the cells; and act as a pH indicator. Further details on these components may be found in Remington's Pharmaceutical Sciences (Maack Publishing Co., Easton, Pa.) hereby incorporated herein by reference in relevant part.

“Perfusion” is used herein in its broadest context to include not only mechanical machine perfusion, but also all means of flushing, washing, bathing, cleaning, diffusing or exposing transplantable biological materials to the compositions described herein. The perfusion may be pulsatile, continuous or irregular in nature.

As used herein, “transplantable biological materials” include, but are not limited to, any mammalian organ, tissue, structure, cell, or membrane, regardless of whether the source is from cadaveric origin, human origin, laboratory origin, or mechanical manufacture. Suitable organs with which the solutions of this invention may be used include, for example, heart, liver, kidney, lungs, pancreas, and small bowel.

The present invention is therefore directed to the use of improved solutions to resurrect or preserve transplantable organs. These solutions alleviate R1 and the concomitant antigenic reactions that result from transplantation. The present invention is therefore further directed towards preventing such toxic events by implementing a defense strategy wherein the toxic substance is either blocked prior to its manufacture, or destroyed before it attacks any transplantable biological material. The compositions utilize a variety of components to address specific aspects of reperfusion injury and antigenic response.

It is contemplated that the additions to the typical University of Wisconsin Solution, which collectively comprise the improved solutions of the present invention, facilitate a reduction in RI and reduce antigenic response. For example, the addition of dexamethasone phosphate in a high dose can be used to prevent Nf kappa b activation of inflammatory mediators, such as tumor necrosis factor (TNF), interleukins, 1, 6, 8 (IL-1, IL-6, IL-8) and NO synthase, as well as adhesion factors which are dependent on the gene activating factor.

In addition to the additional compounds described above, the typical Wisconsin Solution ingredients delineated above can be substituted with other ingredients. For example, adenine and ribose are contemplated as replacements for adenosine. Due to its much longer half-life, oxipurinol is contemplated as a replacement for allopurinol, which may be an ingredient of Wisconsin Solution. Sulfinated starch can replace hydroxyethyl starch. Lactobionate replace gluconate when using the uric acid precursors or derivatives described herein. “Wisconsin Solution” as used herein therefore refers both to the formulation as set forth above, as well as the formulation with the substitutions described herein.

### EXAMPLES

The following examples are intended to illustrate the present invention, and should not be construed as limiting the invention in any way.

### Example 1

The following example was conducted to establish the role of uric acid in protecting against the sequelia associated with diabetic damage. It is established that this sequelia occurs in diabetic patients due to increases in sugar levels that result from either the insufficient production of insulin or the insufficient breakdown of sugar by the insulin, depending of the type of diabetes. Sugar, in excess conditions, can become an oxidizer, which leads to oxidative damage in a patient. Glycosylated hemoglobin (HBA1C) is used to measure the amount of oxidative damage that results from excess sugar in a patient; increased (HBA1C) is the definition used for diabetes. High levels of HBA1C result in, for example, kidney, nerve and heart damage. Uric acid can inhibit production of glycosylated hemoglobin. Chickens were used to confirm this relationship. Chickens are known to have excessively high sugar levels, and should be essentially in a diabetic state. High uric acid prevents the chickens from developing diabetic complications, however; for example, high uric acid levels prevent the elevation of the chicken equivalent to A1C. To confirm the causal relationship between high levels of uric acid and prevention of glycosylation damage, chickens were given allopurinol. This compound specifically converts uric acid back to hypoxanthine. Enough was administered to effect significant conversion of uric acid back to hypoxanthine, evidenced by the lowering of uric acid levels. Once levels of uric acid were reduced to below normal levels, levels of the chicken equivalent of A1C increased dramatically. The chickens then exhibited signs of diabetic complications. This demonstrates that levels of uric acid below normal can lead to increases in compounds that cause oxidative damage.

### Example 2

An 85 year old woman suffering from Alzheimer's for approximately 10 years had a uric acid concentration of 4.5 mg/100 ml blood. 500-1,000 mg of either inosine or hypoxanthine was administered orally per day; her uric acid level was raised to 7.5-8.5 mg/100 ml blood within 14 days. Symptoms, such as not being able to recognize the bathroom, using any stool or chair as a toilet, as well as incontinence and general cognitive decline were observed to be reduced upon raising the uric acid levels.

Three months later, during a routine blood test, it was noticed that the patient's uric acid dropped from 7.5 to 3.5. This drop in uric acid levels can be explained by the disease going into an “acceleration phase” in which the uric acid level is more rapidly depleted.

Six weeks after it was noticed that uric acid levels dropped, the symptoms returned. Additionally, during the “acceleration phase”, twice the usual dose of precursor was needed, i.e. 1,000-2,000 mg/day, to bring the uric acid level back to between 7.5-8.5.

Approximately six weeks after returning the uric acid levels back to above 4.9, the symptoms again were reduced. This demonstrates the correlation of uric acid levels with Alzheimer symptoms, and the ability of the present compositions and methods to treat at least the symptoms of Alzheimer's.

### Example 3

An improved Wisconsin Solution according to the present invention is exemplified by the following components and approximate amounts. The pH of the solution is adjusted to 7.4 with sodium hydroxide or hydrochloric acid as appropriate.

| | | |
|---|---|---|
| Dexamethasone phospate | 100-500 | mg/liter |
| Beta Cyclodextrin hydrate (MW 1135) | 50 | g/liter |
| N-acetylcystein | 10-100 | mm |
| Adenosine monophosphate | 10 | mm |
| Potassium salt of polygalacturonic acid | 100 | mm |
| Allopurinol | 1 | mm |
| D glucose | 10 | mm |
| Calcium Chloride hexahydrate | 1 | mm |
| Sodium urate solution + 7 mg % L-arginine | | |
| Magnesium chloride | 5 | mm |
| Ng monomethyl L-arginine (L-Name) | 200 | mg % |
| Salt | 500,000 | units/liter |
| Potassium dihydrogen phosphate | 25 | mm |
| Polyphenolic substances¹ | | |
| 4 hydroxy tempo | 10 | mm |
| Creatine monohydrate | 5 | g/liter |
| Essential amino acids² | 1-10 | mm |
| Insulin | 50 | units/liter |
| Phenol Red | 12 | mg |

¹extracted from green tea
²histidine, isoleucine, leucine, lysine, methionine, phenylalamine, threonine, tryptophan, and valine

It will be appreciated by those skilled in the art that the actual preferred amounts of the ingredients can be varied according to the specific compound ratio utilized, the particular solutions formulated, and the mode of application. Concentrations for a specific circumstance can be determined using conventional considerations, e.g., by comparisons of the differential activities of the active compounds of this invention with known agents by means of an appropriate conventional pharmacological protocol and extrapolation of the dosages based on the results thereof as is known in the art.

The solutions of the present invention can be used at all temperatures ranging from 0° C. to normal body temperature, 37° C., especially in a temperature range from 4° C. to 8° C. The harvested organ is placed in the chilled solution until it is used for transplant. It is then warmed back to body temperature by gradiated warmer solutions until body temperature is achieved. Perfusion is effected, such as with saline, to remove the solution. Engraftment is then performed.

It will be understood by those skilled in the art that all components in the organ preservation solutions described herein are included in amounts effective to fulfill their described purpose for inclusion. For example, antioxidants are included in an amount effective to inhibit oxygen-derived free radicals; peroxynitrite inhibitors are present in an amount effective to inhibit the formation of peroxynitrite, etc. Thus the “effective amount” of each component in the solution will vary depending on the component. It is within the skill of one practicing in the art to determine the appropriate effective amount for each component.

The individual components of the present solutions are all non-toxic and have been found to be stable during storage. While some of the components of the present solutions are similar to those of other known preservation solutions, it has been found that the addition of certain components described herein can alleviate reperfusion injury and/or reduce the antigenic effect of transplantation in the recipient when compared with the solutions currently known in the art.

The compositions of the present invention are based on a balanced isotonic solution that includes certain electrolytes in physiologically acceptable amounts. Osmolarity of the solutions can be controlled using sodium, potassium, calcium and magnesium ions, as well as glucose and/or sodium bicarbonate.

Whereas particular embodiments of this invention have been described above for purposes of illustration, it will be evident to those skilled in the art that numerous variations of the details of the present invention may be made without departing from the invention as defined in the appended claims.

1. A method for treating an individual having a condition in which uric acid blood concentration level of the individual is below about 4.9 mg/100 ml, comprising:
administering an effective amount of one or more uric acid derivatives and uric acid precursors to the individual to bring the uric acid blood concentration level of the individual above about 4.9 mg/100 ml.

2. The method according to claim 1, further comprising administering one or more antioxidants to the individual.

3. The method according to claim 1, wherein the effective amount of the one or more uric acid derivatives and uric acid precursors comprises a daily dose whereby the uric acid level in the individual is maintained above about 4.9 mg/100 ml and less than 10.0 mg/100 ml.

4. The method according to claim 4, wherein the effective amount of the daily dose of the one or more uric acid derivatives and uric acid precursors is an amount in the range of from 100 mg to 1,000 mg contained in a pharmaceutically acceptable carrier.

5. A unit dose pharmaceutical composition for treating an individual having a condition in which uric acid blood concentration level of the individual is below about 4.9 mg/100 ml, consisting essentially of:
an effective amount of one or more of a derivative of uric acid and a precursor to uric acid sufficient to raise or maintain the uric acid blood concentration level in an individual above about 4.9 mg/100 ml and less than 10.0 mg/100 ml, wherein the effective amount of the one or more uric acid derivatives and uric acid precursors is an amount in the range of from 100 mg to 1,000 mg contained in a pharmaceutically acceptable carrier.

6. A unit dose pharmaceutical composition for treating an individual having a condition in which uric acid blood concentration level of the individual is below about 4.9 mg/100 ml, consisting essentially of:
an effective amount of one or more of a derivative of uric acid and a precursor to uric acid sufficient to raise or maintain the uric acid blood concentration level in an individual above about 4.9 mg/100 ml and less than 10.0 mg/100 ml, wherein the unit dose of the one or more uric acid derivatives and uric acid precursors is an amount in the range of from 100 mg to 1,000 mg contained in a pharmaceutically acceptable carrier, and
one or more antioxidants.

7. A method of preventing oxidative damage to an individual, comprising the steps of:
adjusting the uric acid blood concentration level of the individual to be maintained at or above normal levels by administering a succession of effective amounts of one or more of a derivative of uric acid and a precursor to uric acid whereby uric acid level is greater than about 4.9 mg/ml.

8. The method according to claim 12, wherein the adjusting step comprises administering a daily dose of an effective amount of one or more uric acid derivatives and uric acid precursors to the individual to bring the uric acid blood concentration level of the individual above about 4.9 mg/100 ml and less than 10.0 mg/100 ml, the effective amount effective amount of the daily dose of the one or more uric acid derivatives and uric acid precursors is an amount in the range of from 100 mg to 1,000 mg contained in a pharmaceutically acceptable carrier.

9. A method for prophylactically treating an individual in anticipation of a condition in which uric acid blood concentration level in the individual would be below about 4.9 mg/100 ml, comprising:
administering a daily dose of an effective amount of one or more uric acid derivatives and uric acid precursors to the individual, whereby uric acid level in the individual is maintained greater than about 4.9 mg/100 ml, the effective amount of the daily dose of the one or more uric acid derivatives and uric acid precursors is an amount in the range of from 100 mg to 1,000 mg contained in a pharmaceutically acceptable carrier.

10. The method according to claim 9, wherein the condition is an occurrence of radiation induced oxidative damage.

11. The method according to claim 1, wherein the administering step is further effective to maintain the uric acid blood concentration level of the individual less than about 10.0 mg/100 ml.

12. The method according to claim 7, wherein the adjusting step comprises administering the daily dose of an effective amount of one or more uric acid derivatives and uric acid precursors to the individual to maintain the uric acid blood concentration level of the individual less than about 10.0 mg/100 ml.

13. The method according to claim 9, wherein the administering step is further effective to maintain the uric acid blood concentration level of the individual less than about 10.0 mg/100 ml.

14. The method according to claim 13, wherein the condition is an occurrence of radiation induced oxidative damage.
